(19) United States
(12) Patent Application Publication
(10) Pub. No.: US 2006/0069360 A1
(43) Pub. Date: Mar. 30, 2006

(54) Absorbent article with insult indicators

(75) Inventors: Andrew M. Long, Appleton, WI (US); Christopher P. Olson, Neenah, WI (US); Shirlee A. Weber, Neenah, WI (US)

Correspondence Address: SENNIGER POWERS, ONE METROPOLITAN SQUARE, 16TH FLOOR, ST LOUIS, MO, 63102, US

(73) Assignee: Kimberly-Clark Worldwide, Inc.

(21) Appl. No.: 10/953,138

(22) Filed: Sep. 29, 2004

(51) Int. Cl. A61F 13/15

(52) U.S. Cl. 604/361

(57) ABSTRACT

In a method of toilet training a wearer of a disposable absorbent article, an active graphic visually perceptible from within the interior of the article is activated in response to liquid insult of the article and at least one other signal perceptible from at least one of the interior and the exterior of the article is provided in response to liquid insult of the article. An absorbent article of the present invention generally has a first insult indicator with an active graphic visually perceptible from the inner surface of the article that is responsive to a liquid insult of the absorbent article to provide a signal perceptible from the inner surface of the article. A second insult indicator is responsive to the liquid insult of the absorbent article to provide a signal perceptible from at least one of the inner surface of the article and exterior of the article.

### FIELD OF THE INVENTION

The present invention relates generally to absorbent articles intended for personal wear, and more particularly to such an absorbent article used in toilet training of children.

### BACKGROUND OF THE INVENTION

Disposable absorbent articles find widespread use as personal care products such as diapers, children's toilet training pants and other infant and child care products, adult incontinence garments and other adult care products, sanitary napkins and other feminine care products and the like, as well as surgical bandages and sponges. These articles absorb and contain body waste and are intended to be discarded after a limited period of use; i.e., the articles are not intended to be laundered or otherwise restored for reuse. Conventional disposable absorbent articles comprise an absorbent body disposed between an inner layer adapted for contacting the wearer's skin and an outer layer for inhibiting liquid waste absorbed by the absorbent body from leaking out of the article. The inner layer of the absorbent article is typically liquid permeable to permit body waste to pass therethrough for absorption by the absorbent body.

Disposable absorbent training pants, in particular, are useful in toilet training children. Typically, these disposable undergarments are similar to washable, cloth underwear in the manner in which they are put on and worn, yet provide an absorbent function similar to diapers to maintain skin health. Training pants provide a child undergoing toilet training with an undergarment that eases the transition from diapers to washable, cloth underwear as they become more confident in their ability to use the toilet independently.

In order to learn to use the toilet independently, a child must first learn to recognize when urination has occurred. Because urination may often occur during an activity that distracts the child to the extent that the child does not notice urination, this recognition can represent a substantial hurdle in the training process. Also, a child's ability to recognize when urination occurs may be hampered by the improved performance of disposable absorbent undergarments which quickly draw and retain urine away from the wearer's skin after an insult occurs.

Close monitoring of a toilet-training child by a caregiver can be helpful in that when urination occurs it can be discussed by the child and caregiver to enhance and improve the learning experience. Therefore, it is beneficial to provide the caregiver with immediate notification and/or verification that urination has occurred so that it may be discussed with the child while the event is still fresh in the child's mind.

Several attempts have been made at improving toilet training aids for toilet training pants. For example, training pants that include a temperature change member and/or a dimensional change member which provide a temperature or pressure change sensation to alert the child wearing the pants that urination has occurred have been disclosed. Additional training aids have been used to alert the caregiver and/or child that urination has occurred. Such training aids include disappearing graphics disposed on the outer cover of the pants, audible alarms, vibration sensors, and light indicators that may provide visual or other sensory indication of urination.

Existing training pants having one or more training aids that alert only the wearer, or only the caregiver, to an insult of the pants promote prolonged debates between the child and the caregiver as to whether an accident has occurred. One of the first obstacles to successful toilet training is ending the deniability of the occurrence of an accident by the child. There is a need, therefore, to provide a suitable absorbent article that enhances the toilet training experience.

### SUMMARY OF THE INVENTION

In one embodiment of the present invention, a method of toilet training a wearer of a disposable absorbent generally comprises activating an active graphic in response to liquid insult of the article. The active graphic is visually perceptible from within the interior of the article. At least one other signal is provided in response to liquid insult of the article. The at least one other signal being perceptible from at least one of the interior and the exterior of the article.

In one embodiment of an absorbent article of the present invention, the absorbent article for toilet training a wearer of the article has an inner surface that faces the article wearer and an outer surface opposite the inner surface. The article comprises an outer cover at least in part defining the outer surface of the article, a liner in opposed relationship with the outer cover and at least in part defining the inner surface of the article, and an absorbent structure disposed between the liner and the outer cover. A first insult indicator is responsive to a liquid insult of the absorbent article to provide a signal perceptible from the inner surface of the article. The first insult indicator has an active graphic visually perceptible. from the inner surface of the article that is activated in response to liquid insult of the article. A second insult indicator is responsive to the liquid insult of the absorbent article to provide a signal perceptible from at least one of the inner surface of the article and exterior of the article.

In another embodiment, the absorbent article for toilet training a wearer of the article generally comprises an outer cover at least in part defining the outer surface of the article, a liner in opposed relationship with the outer cover and at least in part defining the inner surface of the article, and an absorbent structure disposed between the liner and the outer cover. A first insult indicator is responsive to a liquid insult of the absorbent article to provide a non-tactile signal perceptible from the inner surface of the article. A second insult indicator is responsive to the liquid insult of the absorbent article to provide a tactile sensation to the wearer.

Other features of the invention will be in part apparent and in part pointed out hereinafter.

### BRIEF DESCRIPTION OF THE DRAWINGS

FIG. 1 is a side perspective of an article of the present invention shown in the form of a pair of training pants having a mechanical fastening system fastened on one side of the training pants and unfastened on the opposite side thereof;

FIG. 2 is a bottom plan view of the training pants of FIG. 1 with the pants in an unfastened, unfolded and laid flat condition, and showing the surface of the training pants that faces away from the wearer;

FIG. 3 is a top plan view similar to FIG. 2 showing the inner surface of the training pants that faces the wearer when worn and with portions cut away to show underlying features;

FIG. 4 is a section view taken along the plane including line 4-4 of FIG. 3 showing the placement of a graphic visible from the inner surface of the article;

FIG. 4A is a section view similar to FIG. 4 but showing an alternative embodiment of the training pants;

FIG. 5 is a plan view similar to FIG. 3 but showing an alternative embodiment of the training pants;

FIG. 5A is a section view taken along the plane including line 5A-5A of FIG. 5;

FIG. 6 is an enlarged schematic front elevation of a wetness indicator of FIG. 5 with portions partially broken away to reveal internal construction;

FIG. 7 is a section view taken along the plane including line 7-7 of FIG. 6;

FIG. 8 is cross-section similar to FIG. 7 but showing an alternative second embodiment of a wetness indicator including temperature change agent;

FIG. 9 illustrates a partial cross-section similar to FIG. 4 but showing an alternative embodiment of the training pants including an inflatable container;

FIG. 10 is a view similar to FIG. 9 but showing a foaming agent present in the inflatable container;

FIG. 11 is a view similar to FIG. 9 but showing training pants having an external wetness indicator mounted thereon;

FIG. 12 is a top plan view of the embodiment of FIG. 11 showing the inner surface of the training pants that faces the wearer when worn and with portions cut away to show underlying features; and

FIG. 13 is a view similar to FIG. 11 but showing training pants having an alternative embodiment of an external wetness indicator.

Corresponding reference characters indicate corresponding parts throughout the drawings.

### DETAILED DESCRIPTION

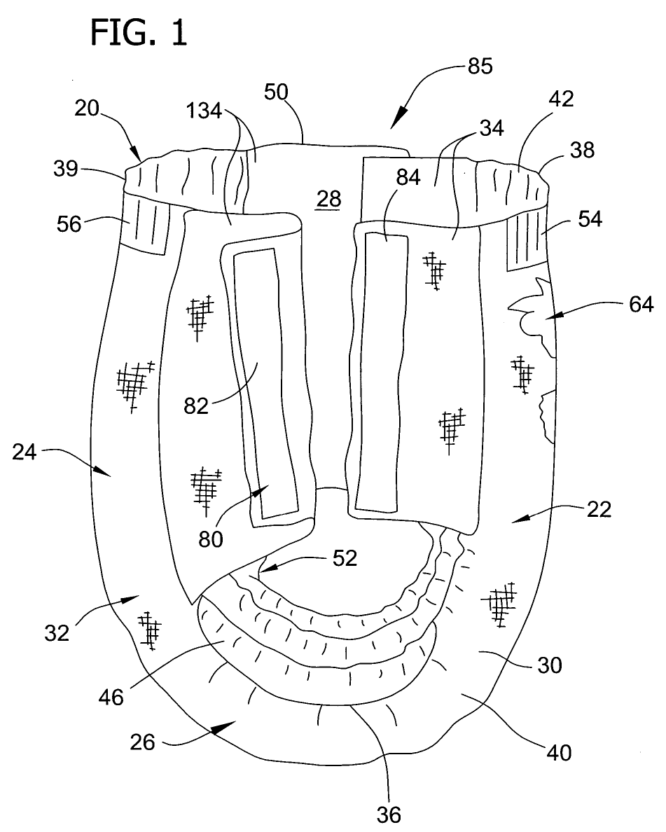

Referring now to the drawings and in particular to FIG. 1, an absorbent article of the present invention is representatively illustrated therein in the form of children's toilet training pants and is indicated in its entirety by the reference numeral 20. The absorbent article 20 may or may not be disposable, which refers to articles that are intended to be discarded after a limited period of use instead of being laundered or otherwise conditioned for reuse. It is understood that the present invention is suitable for use with various other absorbent articles intended for personal wear, including but not limited to diapers, feminine hygiene products, incontinence products, medical garments, surgical pads and bandages, other personal care or health care garments, and the like without departing from the scope of the present invention.

By way of illustration only, various materials and methods for constructing training pants such as the pants 20 of the various aspects of the present invention are disclosed in PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al. which are incorporated herein by reference.

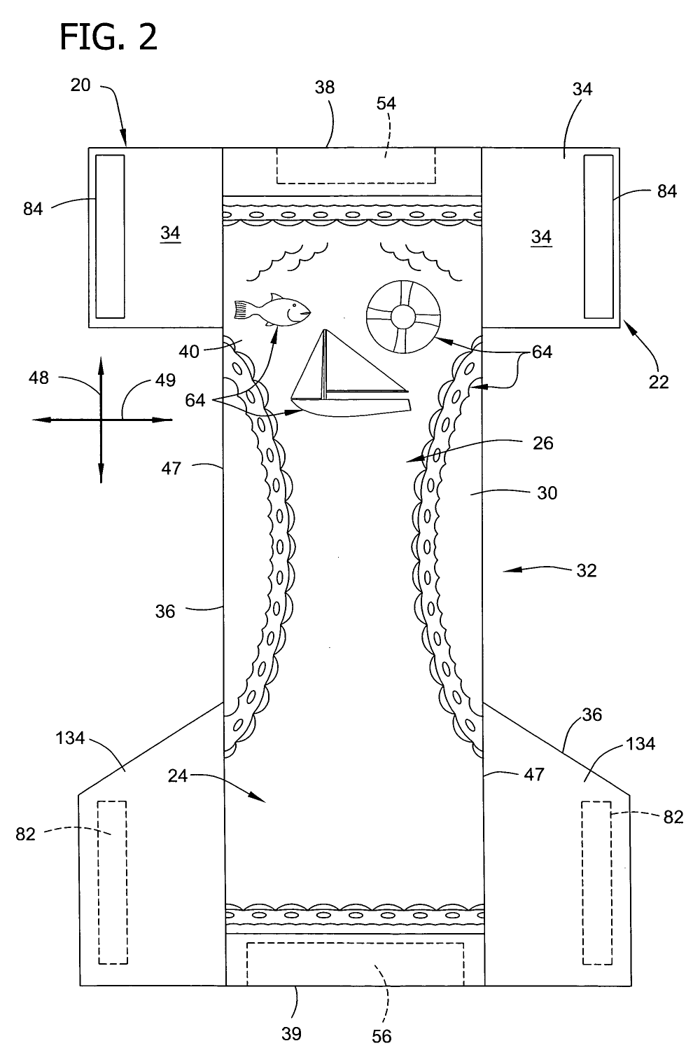

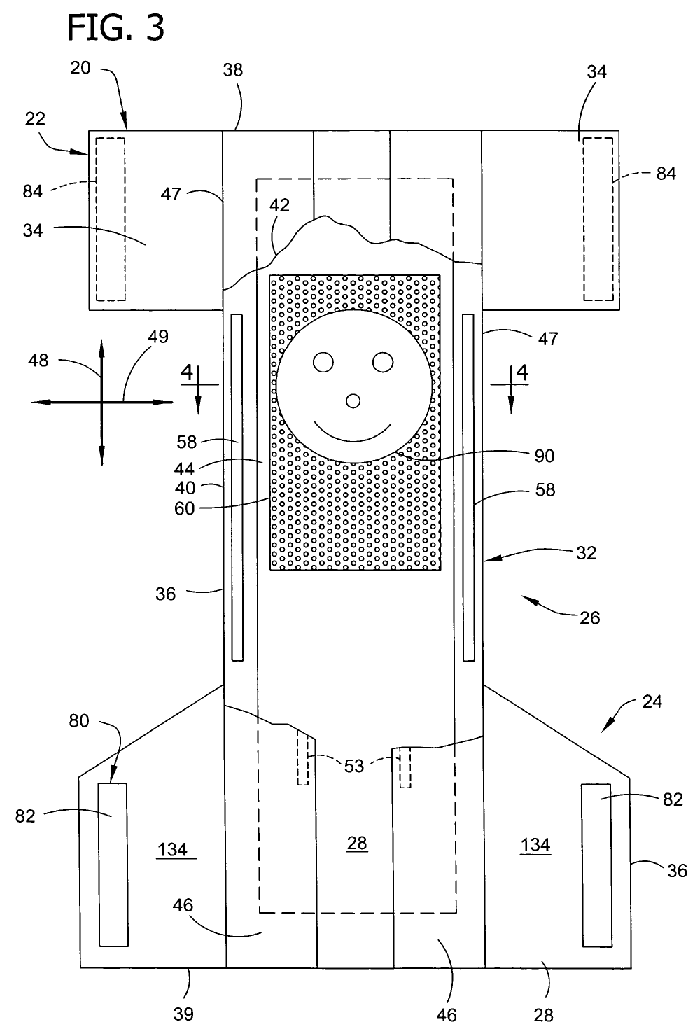

The pair of training pants 20 is illustrated in FIG. 1 in a partially fastened condition. The pants 20 define a longitudinal direction 48 and a lateral direction 49 perpendicular to the longitudinal direction as shown in FIGS. 2 and 3. The pants 20 further define a pair of longitudinal end regions, otherwise referred to herein as a front waist region 22 and a back waist region 24, and a center region, otherwise referred to herein as a crotch region 26, extending longitudinally between and interconnecting the front and back waist regions 22, 24. The front and back waist regions 22, 24 include those portions of the pants 20, which when worn, wholly or partially cover or encircle the waist or mid-lower torso of the wearer. The crotch region 26 generally is that portion of the pants 20 which, when worn, is positioned between the legs of the wearer and covers the lower torso and crotch of the wearer. The pants 20 also define an inner surface 28 adapted in use (e.g., positioned relative to the other components of the pants 20) to be disposed toward the wearer, and an outer surface 30 opposite the inner surface. With additional reference to FIGS. 2 and 3, the pair of training pants 20 has a pair of laterally opposite side edges 36 and a pair of longitudinally opposite waist edges (broadly, longitudinal ends), respectively designated front waist edge 38 and back waist edge 39.

The illustrated pants 20 include an absorbent assembly, generally indicated at 32, and a fastening system for securing the pants in a three-dimensional pants configuration. In the aspect of FIGS. 1-3, the training pants 20 include a generally rectangular central absorbent assembly 32 and side panels 34, 134 formed separately from and secured to the central absorbent assembly. The side panels 34, 134 are permanently bonded along seams 66 to the central absorbent assembly 32 in the respective front and back waist regions 22 and 24 of the pants 20. More particularly, the front side panels 34 can be permanently bonded to and extend transversely outward beyond side margins 47 of the absorbent assembly 32 at the front waist region 22, and the back side panels 134 can be permanently bonded to and extend transversely outward beyond the side margins of the absorbent assembly at the back waist region 24. The side panels 34 and 134 may be bonded to the absorbent assembly 32 using attachment means known to those skilled in the art such as adhesive, thermal or ultrasonic bonding.

The front and back side panels 34 and 134, upon wearing of the pants 20, thus include the portions of the training pants 20 which are positioned on the hips of the wearer. The front and back side panels 34 and 134 can be permanently bonded together to form the three-dimensional configuration of the pants 20, or be releasably connected with one another such as by the fastening system 80 of the illustrated aspects. Suitable elastic materials, as well as one process of incorporating elastic side panels into training pants, are described in the following U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference in their entirety to the extent they are consistent (i.e., not in conflict) herewith. In particular aspects, the elastic material may include a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT application WO 01/88245 in the name of Welch et al.; all of which are incorporated herein by reference in their entirety to the extent they are consistent (i.e., not in conflict) herewith. As is known in the art, the side panels 34, 134 may include elastic material or stretchable but inelastic materials.

The absorbent assembly 32 is illustrated in FIGS. 1-3 as having a rectangular shape. However, it is contemplated that the absorbent assembly 32 may have other shapes (e.g., hourglass, T-shaped, I-shaped, and the like) without departing from the scope of this invention. It is also understood that the side panels 34, 134 may instead be formed integrally with the absorbent assembly 32 without departing from the scope of this invention. In such a configuration, the side panels 34 and 134 and the absorbent assembly would include at least some common materials, such as the bodyside liner 42, outercover 40, other materials and/or combinations thereof.

The absorbent assembly 32 includes an outer cover 40 and a bodyside liner 42 (FIGS. 1 and 3) in a superposed (opposed) relation therewith. The liner 42 can be suitably joined to the outer cover 40 along at least a portion of the longitudinal ends of the pants 20. The liner 42 can be suitably adapted, i.e., positioned relative to the other components of the pants 20, for contiguous relationship with the wearer's skin during wear of the pants. The absorbent assembly 32 also includes an absorbent structure 44 (FIG. 3) disposed between the outer cover 40 and the bodyside liner 42 for absorbing liquid body exudates exuded by the wearer.

With the training pants 20 in the fastened position as partially illustrated in FIG. 1, the front and back waist regions are connected together by the fastening system 80 to define the three-dimensional pants configuration having a waist opening 50 and a pair of leg openings 52. The front and back waist edges 38 and 39 (e.g., longitudinal ends) of the training pants 20 are configured to encircle the waist of the wearer to define the waist opening 50 (FIG. 1) of the pants.

The pants 20 may further include a pair of containment flaps 46 for inhibiting the lateral flow of body exudates. As illustrated in FIG. 3, the containment flaps 46 can be operatively attached to the pants 20 in any suitable manner as is well known in the art. In particular, suitable constructions and arrangements for the containment flaps 46 are generally well known to those skilled in the art and are described in U.S. Pat. No. 4,704,116 issued Nov. 3, 1987 to Enloe, which is incorporated herein by reference.

To further enhance containment and/or absorption of body exudates, the training pants 20 may include a front waist elastic member 54 (FIG. 2), a rear waist elastic member 56, and leg elastic members 58 (FIG. 3), as are known to those skilled in the art. The flap elastic members 53, the waist elastic members 54 and 56, and the leg elastic members 58 can be formed of any suitable elastic material that is well known to those skilled in the art.

The front and back side panels 34 and 134 can be permanently bonded together or be releasably connected with one another such as by the fastening system 80 of the illustrated aspect. The illustrated fastening system 80 includes laterally opposite first fastening components 82 adapted for refastenable engagement to corresponding laterally opposite second fastening components 84. In one aspect, a front or outer surface of each of the fastening components 82, 84 includes a plurality of engaging elements. The engaging elements of the first fastening components 82 are adapted to repeatedly engage and disengage corresponding engaging elements of the second fastening components 84 to releasably secure the pants 20 in its three-dimensional configuration. The fastening components 82, 84 can include any refastenable fasteners suitable for absorbent articles, such as adhesive fasteners, cohesive fasteners, mechanical fasteners, or the like. Suitable fastening systems are also disclosed in the previously incorporated PCT Patent Application WO 00/37009 published Jun. 29, 2000 by A. Fletcher et al. and the previously incorporated U.S. Pat. No. 6,645,190 issued Nov. 11, 2003 to Olson et al.

The outer cover 40 may suitably include a material that is substantially liquid impermeable. The outer cover 40 may be provided by a single layer of liquid impermeable material, or more suitably include a multi-layered laminate structure in which at least one of the layers is liquid impermeable. In particular aspects, the outer layer may suitably provides a relatively cloth-like texture to the wearer. A suitable liquid impermeable film for use as a liquid impermeable inner layer, or a single layer liquid impermeable outercover 40 is a 0.025 millimeter (1.0 mil) polyethylene film commercially available from Edison Plastics Company of South Plainfield, N.J. Alternatively, the outer cover 40 may include a woven or non-woven fibrous web layer that has been totally or partially constructed or treated to impart the desired levels of liquid impermeability to selected regions that are adjacent or proximate the absorbent structure. The outer cover 40 may also be stretchable, and in some aspects it may be elastomeric. Reference is made to U.S. Pat. No. 5,883,028, issued to Morman et al., U.S. Pat. No. 5,116,662 issued to Morman and U.S. Pat. No. 5,114,781 issued to Morman, all of which are hereby incorporated herein by reference, for additional information regarding suitable outer cover materials.

The bodyside liner 42 is suitably compliant, soft-feeling, and non-irritating to the wearer's skin. The bodyside liner 42 is also sufficiently liquid permeable to permit liquid body exudates to readily penetrate through its thickness to the absorbent structure 44. A suitable liquid permeable bodyside liner 42 is a nonwoven polyethylene/polypropylene bicomponent web having a basis weight of about 27 gsm; the web may be spunbonded or a bonded carded web. Alternatively, the bodyside liner 42 may also be stretchable, and in some aspects it may be elastomeric. Reference is made to U.S. patent application Ser. No. 09/563,417 filed on May 3, 2000 by Roessler et al., U.S. patent application Ser. No. 09/698,512 filed on Oct. 27, 2000 by Vukos et al., both of which are incorporated by reference herein, for additional information regarding bodyside liner material.

An absorbent structure 44 may be disposed between the outer cover 40 and the bodyside liner 42, which can be joined together by any suitable means such as adhesives, ultrasonic bonds, thermal bonds, or the like. While the illustrated absorbent structure 44 is shown and described herein as extending from the crotch region 26 into both the front and back waist regions 22 and 24, it is contemplated that the absorbent structure may extend from the crotch region into only the front waist region, or only the back waist region, without departing from the scope of this invention.

The absorbent structure 44 can be suitably compressible, conformable, non-irritating to a wearer's skin, and capable of absorbing and retaining liquids and certain body wastes. For example, the absorbent structure 44 may include cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof.

The materials may be formed into an absorbent web structure by employing various conventional methods and techniques known in the art. For example, the absorbent structure 44 may be formed by a dry-forming technique, an air forming technique, a wet-forming technique, a foam-forming technique, or the like, as well as combinations thereof. Methods and apparatus for carrying out such techniques are well known in the art. The absorbent structure 44 may alternatively include a coform material such as the material disclosed in U.S. Pat. No. 4,100,324 to Anderson, et al.; U.S. Pat. No. 5,284,703 to Everhart, et al.; and U.S. Pat. No. 5,350,624 to Georger, et al.; which are incorporated herein by reference.

The term “coform material” generally refers to composite materials comprising a mixture or stabilized matrix of thermoplastic fibers and a second non-thermoplastic material. As an example, coform materials are made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic absorbent materials, treated polymeric staple fibers and the like. Any of a variety of synthetic polymers may be utilized as the melt-spun component of the coform material. For instance, in certain aspects, thermoplastic polymers can be utilized. Some examples of suitable thermoplastics that can be utilized include polyolefins, such as polyethylene, polypropylene, polybutylene and the like; polyamides; and polyesters. In one aspect, the thermoplastic polymer is polypropylene.

Superabsorbent material may be suitably present in the absorbent structure 44 in an amount of from about 0 to about 90 weight percent based on total weight of the absorbent structure. The absorbent structure 44 may suitably have a density within the range of about 0.10 to about 0.35 grams per cubic centimeter. Superabsorbent materials are well known in the art and can be selected from natural, synthetic, and modified natural polymers and materials.

In one aspect, the absorbent structure 44 may be stretchable so as not to inhibit the stretchability of other components to which the absorbent structure may be adhered, such as the outer cover 40 and bodyside liner 42. For example, the absorbent structure may include materials disclosed in U.S. Pat. Nos. 5,964,743, 5,645,542, 6,231,557, 6,362,389, and international patent application WO 03/051254, the disclosure of each of which is incorporated by reference herein.

In some aspects, a surge management layer (not shown) may be located adjacent the absorbent structure 44 (e.g., between the absorbent structure and the liner 42 ) and attached to various components of the article 20 such as the absorbent structure and/or the bodyside liner 42 by methods known in the art, such as by adhesive, ultrasonic or thermal bonding. A surge management layer helps to decelerate and diffuse surges or gushes of liquid that may be rapidly introduced into the absorbent structure 44 of the article 20. Desirably, the surge management layer can rapidly accept and temporarily hold the liquid prior to releasing the liquid into the storage or retention portions of the absorbent structure 44. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are incorporated by reference herein.

Optionally, a substantially liquid permeable wrapsheet (not shown) may surround the absorbent structure 44 to help maintain the integrity of the absorbent structure 44.

The training pants 20 of the present invention include at least two insult indicators (sometimes otherwise referred to as training aids or training elements) that in combination provide a synergistic toilet training experience. For example, in one embodiment the training pants comprises at least one insult indicator that provides a tactile signal to the wearer in response to a liquid insult of the pants and at least one other insult indicator that provides a non-tactile signal that is perceptible to the wearer and/or to the caregiver.

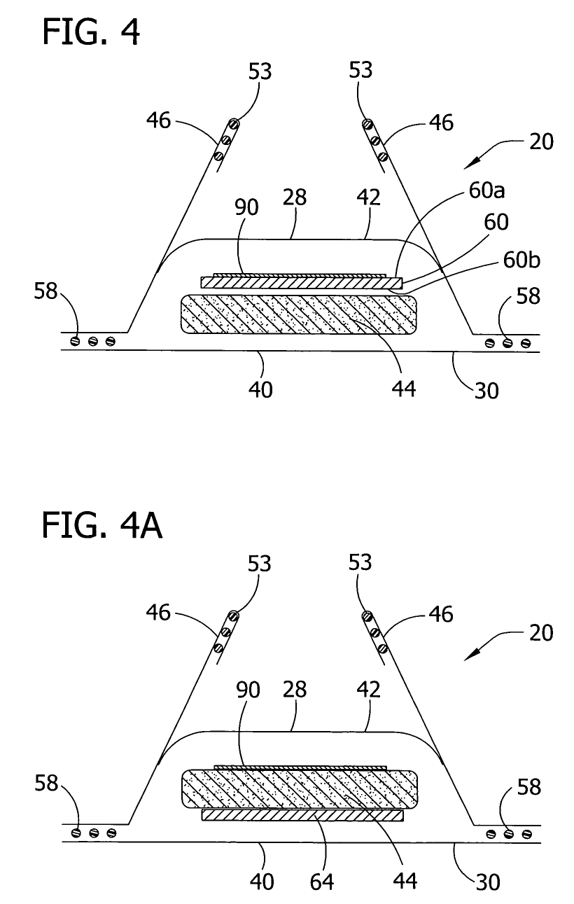

In one particularly suitable embodiment, shown in FIGS. 3 and 4, a first insult indicator in the form of a wetness indicator 60 provides a tactile signal, and in particular a prolonged feeling of wetness against the wearer's skin, in response to a liquid insult of the pants 20. The wetness indicator 60 of the illustrated embodiment comprises an absorbent pledget disposed between the bodyside liner 42 and the absorbent structure 44 of the training pants 20. The pledget 60 is placed in the crotch region 26 of the article 20 in a position where it is most likely to be contacted with urine. When the pledget 60 is contacted by liquid (e.g., urine) it stays wet for a prolonged duration and communicates a wet feeling to the wearer's skin to indicate to the wearer that an insult has occurred. In one embodiment, the wetness feeling from the pledget 60 may last approximately 15 minutes, after which the liquid is drawn into the absorbent structure 44. The temporary wetness of the pledget 60 acting against the wearer's skin provides a tactile signal of wetness to the wearer without jeopardizing the skin health of the wearer.

In a particularly suitable aspect, the pledget 60 may comprise a coform material having a fluff ratio of at least 65:35 fluff/staple fiber that is apertured and covered with a 0.6 osy (20.4 gsm) highly wettable spunbond material. The coform material resists collapsing of the pledget upon liquid insult (i.e., wetting) to thereby maintain open void space within the pledget. The coform material suitably has a density in the range of approximately 0.15 g/cc to approximately 0.5 g/cc. The pledget 60 contains little or no superabsorbent material so that the pledget initially takes in liquid body exudates and retains the liquid for a period of time before the liquid is absorbed by the absorbent structure 44 that may contain a higher amount of superabsorbent particles. The pledget cover material may have a colored tint (e.g., blue) to give a noticeable 3-dimensional appearance to the pledget 60. It is contemplated that the pledget 60 may comprise only the coform material, e.g., without a cover, or that the cover may be composed of other materials and/or colors without departing from the scope of this invention.

In a particular embodiment, the pledget may be sized approximately 6 in. (152 mm) by 2.5 in. (64 mm) and placed approximately 3.75 in. (95 mm) down from the front longitudinal edge margin of the pant 20 and 2.25 in. (57 mm) down from the front longitudinal edge margin of the absorbent structure 44. However, it is understood that the pledget may have other sizes and shapes and be otherwise located in the pants 20.

In use, when the wearer of the training pants 20 urinates therein, urine penetrates the liner 42 and is received by the pledget 60. Because there is little or no superabsorbent material in the pledget 60 a relatively high volume of unabsorbed urine is entrained in the interstitial spacing within the pledget material for a prolonged duration before flowing to the absorbent structure 44. The prolonged maintenance of unabsorbed urine in the pledget 60 facilitates the flow of urine from the pledget 60 back through the liner 42 to provide a prolonged feeling of wetness against the wearer's skin for indicating to the wearer that he or she has urinated. In particular, the pledget 60 suitably has a relative surface moisture value of at least about 60 percent at approximately 1 minute after a liquid insult, and more particularly at least about 75 percent at approximately 1 minute after a liquid insult for improved performance.

The relative surface moisture of the pledget 60 as refered to herein is calculated from measurements made using a Surface Dryness Measuring Equipment apparatus manufactured by Hoechst Atkiengesellschaft of West Germany. A detailed description of this type of equipment and its operation can be found in U.S. Pat. No. 4,924,084 issued May 8, 1984 to Lask et al., which is incorporated herein by reference in its entirety. The equipment for this apparatus includes a Strip chart recorder from the Linear Instrument Corporation of Reno, Nev. (Model 1201). The chart recorder records moisture readings from an optical light sensor which in turn is connected to a DC power source. Prior to the conductance of testing, the equipment is turned on and allowed to warm up for a minimum of 45 minutes.

To prepare a sample for testing, a pledget 60 is centered on an absorbent mat. The absorbent mat measures 40.6 by 9.5 centimeters and includes an absorbent structure covered by a liquid permeable liner. The liner is a spunbond polypropylene web having a basis weight of 20 grams per square meter (gsm) and including 0.3 weight percent hydrophilizing surfactant treatment. The absorbent structure includes 16 g of wood pulp fluff and a generally layered arrangement of 12 g of superabsorbent material, and has a density of 0.15 to 0.2 grams per cubic centimeter (g/cc). The absorbent mat is positioned in a leak-proof container. The pledget 60 is positioned so that any attachment means bonds the pad to the liner of the absorbent mat.

To test each sample, each sample is placed on top of a plexiglass plate approximately the same size as that of the sample. In order to normalize the moisture values for each sample, a dry reading and a wet reading are both obtained in addition to the actual wetness curve which is generated over a preselected time interval of 10 minutes.

To obtain a dry reading and thus a lower limit on the graph, the sensor is placed over the top of the pledget 60 with the longitudinal axis of the sample and with the ends of the optical light sensor extending equidistant over both side edges of the sample. The sample is positioned with the pledget 60 adjacent the light sensor and the leak-proof container on the plexiglass support. The chart pen is then activated by switching the recorder from stand-by to record and the pen is then zeroed over the 20 grid mark location. The recorder is then returned to stand-by and the detector is removed from the sample.

Next a stainless steel ring having a 6 centimeter inner diameter, a height of 4 centimeters and a weight of approximately 326 grams is centered over the pledget 60 in the same location as the previous dry reading. Into the center of the steel ring there is poured 80 milliliters of certified blood bank saline (Catalogue No. B3158-1) from the Baxter Healthcare Corporation, Scientific Products Division, McGraw Park, Ill. The saline solution is a stabilized isotonic 0.9% saline solution containing no preservatives. The saline solution is at ambient temperature (72° to 74° F.) (22° to 23° C.). The 80 milliliters of saline solution is quickly poured into the ring and thus onto the pledget 60. Immediately after the saline solution is absorbed below the surface of the pad (no excess liquid standing on the pad), the stainless steel ring is removed and the optical light sensor is immediately placed on top of the sample in the same manner as described before and the chart recorder is switched from stand-by to record. The recorder is adjusted to a chart speed of 1 centimeter per minute and the test is allowed to run for a total of 10 minutes.

At the end of the ten minute interval, the chart pen is lifted and the chart is turned off by switching the chart to stand-by. Next, the ring is placed back on top of the sample in the same location as before and the sample is totally saturated by pouring an additional quantity of saline solution generally in an amount of about 100 milliliters so as to completely saturate the absorbent mat. The amount of liquid in the absorbent mat after the second insult should be enough such that the weight of the sensor causes slight flow back of the liquid to the surface. The ring is then removed and the optical light sensor, whose optical sensing portion is wiped free of any excess saline solution from the previous measurement, is placed in the same location on top of the sample in the same manner as described above. The chart is again switched from stand-by to record and the chart is either momentarily activated or the chart paper is moved back and forth so as to achieve a mark or location on the grid paper representing the total saturation measurement for the sample. Each sample tested then has a zero or dry value (V_{D}), a total saturation value (V_{S}) and a time dependent curve extending from the point of absorption of the initial 80 milliliters of saline solution to a point 10 minutes later.

Following the collection of this data, the relative surface moisture values are calculated using the following equation:

$$relative surface moisture ( \% ) = \frac{{V}_{T} - {V}_{D}}{{V}_{S} - {V}_{D}} \times 100 = {V}_{R}$$

Where:
V_{T} is the value on the curve at a given time.
V_{D} is the value on the curve when the sample is dry.
V_{S} is the value on the curve when the sample is saturated.

Still referring to FIGS. 3 and 4, a second insult indicator in the form of interior active graphics 90 provides a non-tactile signal within the interior of the pants 20 in response to a liquid insult thereof. The interior active graphics 90 suitably provide a signal that is perceptible, i.e., visible by both the caregiver and the wearer of the article to confirm that a liquid insult has occurred. As used herein, the term “active graphic” refers to a graphic that visibly (to an unaided human eye) changes appearance in response to a liquid insult of the article. For example, the active graphic may fade or otherwise appear faint following liquid insult (often referred to as a fading graphic), or the active graphic may change from a relatively faint appearance to a bolder, or at least more readily visible appearance (often referred to as an appearing graphic), or the active graphic may change colors in response to a liquid insult.

In the illustrated embodiment of FIGS. 3 and 4, the interior active graphic 90 is suitably a fading graphic, for example in the form of a smiley face that is visible from the inner surface 28 of the article 20. For instance, the graphic 90 can be a layer of ink suitably disposed below the bodyside liner 42 (e.g., away from the wearer) so that the ink is not readily transferred from the article to the skin of the wearer. More particularly, as shown in FIG. 4, the graphic 90 is printed on the top surface 60 a of the pledget 60 so that the graphic is visible through the liner 42 from the inner surface 28 of the article 20. However, it is contemplated that the active graphic 90 may be placed at other locations, or at multiple locations, within the interior of the pants 20 (e.g., on the liner 42, on the bottom surface 60 b of pledget, on the absorbent structure 44, etc.). The graphic may be suitably applied using a fiexographic printing process. Flexographic printing apparatus are known to those skilled in art. For example such apparatus are shown and/or described in U.S. Pat. No. 5,458,590 (Schleinz et al.); U.S. Pat. No. 5,566,616 (Schleinz et al.); U.S. 2003/0019374A1 (Harte); and U.S. Pat. No. 4,896,600 (Rogge et al.). Alternatively, the active graphic 90 may be printed, sprayed, or otherwise applied to the absorbent article 20 by another suitable printing method (e.g., ink jet, rotogravure, etc.).

In one aspect, the fading graphic may be formed from an ink that is soluble in aqueous solutions such as urine. As such, the ink can be positioned in the pants 20 so that it becomes wet and dissolves when the product is insulted with liquid. Suitable urine-soluble inks are available from a variety of commercial vendors, such as Sun Chemical Corp. of Philadelphia, Pa., USA under the trade designation AQUA DESTRUCT. Particular urine-soluble compositions are disclosed in U.S. Pat. No. 4,022,211 issued May 10, 1977 to Timmons et al., which is incorporated herein by reference to the extent it is consistent (i.e., not in conflict) herewith. The ink color can be selected to provide a pleasing appearance and graphic impact, including fading rapidly upon contact with liquid. In particular aspects, and to facilitate rapid fading, the fading graphics can comprise line drawings having a line width of from about 1 to about 5 millimeters.

The interior active graphic 90 may include, but is not limited to, scenes, characters, animals, objects, alphanumerics such as numbers, letters, words, phrases and the like. In particular aspects, the graphic 90 may also be gender specific, that is, the graphic may be generally considered to be of interest to boys or to girls. It is also contemplated that the interior active graphic 90 may include portions thereof which are inactive, or permanent, as long as other portions of the graphic are active in response to liquid insult. It is also understood that more than one interior active graphic 90 may be located within the pants 20 without departing from the scope of the invention. Further reference is be made to U.S. patent application Ser. No. 10/748,411 filed Dec. 29, 2003 and U.S. patent application Ser. No. 10/881,255 filed Jun. 30, 2004, the disclosures of both of which are incorporated by reference herein, for additional information regarding the placement, composition, and other details and alternatives of the active graphic 90.

In use, the graphic 90 (i.e., the non-tactile insult indicator) visible to wearer of the article 20 from the inner surface 28 acts in concert with the wetness indicator 60 (i.e., the tactile insult indicator) to assist in toilet training of the wearer. For example, the child comes to recognize that the active graphic 90 changes in appearance whenever the wet feeling against the skin is felt. This encourages the child to pull the pants 20 up and down to view the inner surface 28 of the article 20 and inspect the graphic 90, an activity which is a key to toilet training and requires a relatively high level of coordination. In addition, as can be readily appreciated a graphic 90 may encourage the wearer to refrain from wetting the article 20 in order to keep the graphic in place. Further, the graphic 90 can provide the wearer with a feeling of “ownership” over the graphic, as the graphic is intended primarily for their viewing. Still further, an active graphic 90 positioned to be viewed from the inner surface 28 of the article 20 is more sensitive to even small insults than active graphics otherwise located due to their proximity to the target area. As such, interior active graphics can be particularly effective in late stage training.

The interior active graphic 90 also provides the caregiver with verifiable evidence that liquid insult has occurred, e.g., without having to touch the wet liner of the pants 20, which can be used to prevent prolonged debates between the child and caregiver on whether an insult has occurred. The interior active graphic 90 also provides the caregiver with a means for relating to the child the relationship between the act of urinating and both the changing of the graphic and wet feeling in the child's pants 20, thereby enhancing the emotional aspects of toilet training.

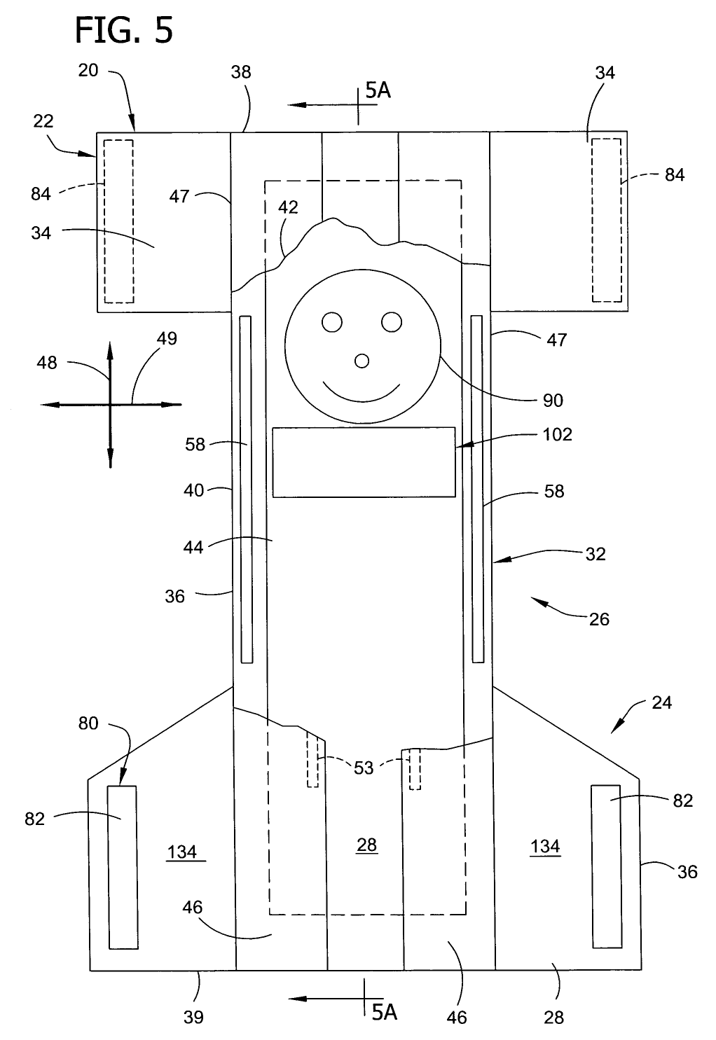

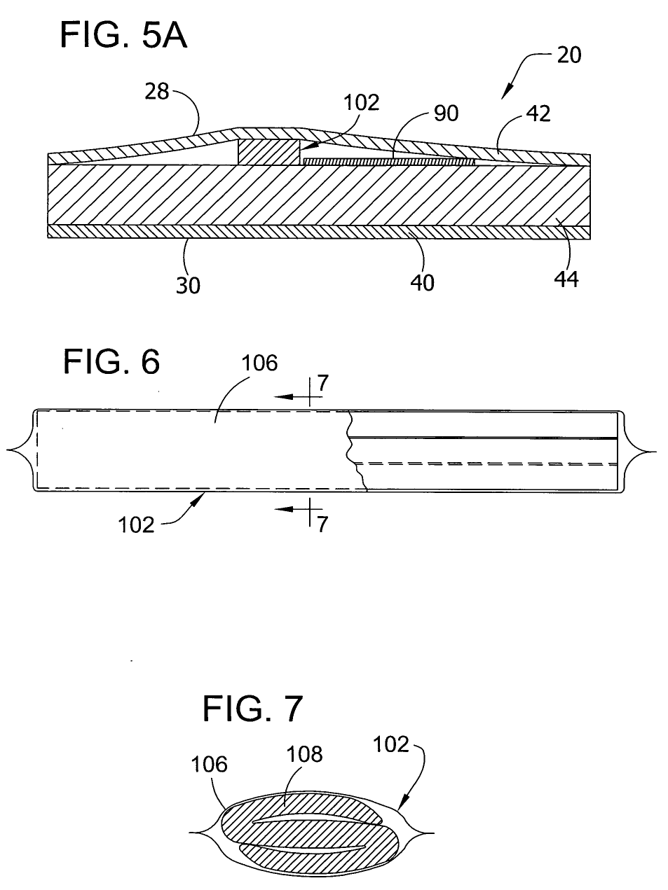

It is understood that insult indicators other than the wetness indicator 60 of FIGS. 3 and 4 may suitably provide a tactile signal to the wearer in response to liquid insult of the pants and remain within the scope of the present invention. For example, FIGS. 5-7 illustrate an alternative embodiment of the training pants 20 including a non-tactile insult indicator in the form of an interior active graphic 90 and a tactile insult indicator in the form of a wetness indicator 102 comprising a liquid permeable enclosure 106 having a liquid absorbent body 108 enclosed within the interior of the enclosure. In one embodiment, the absorbent body 108 of the wetness indicator 102 is a “fan-folded” structure that comprises a sheet material folded over upon itself at least once to form at least two layers that enhances the strength of the absorbent body since each fold layer will act to support adjacent fold layers, further magnifying the expansive pressure within the enclosure 106. Reference is made to co-assigned U.S. patent Ser. No. 10/038,863, filed Dec. 31, 2001 by Olson, et al. and entitled WETNESS INDICATOR FOR ALERTING A WEARER TO URINATION and Ser. No. 10/022,328, filed Dec. 31, 2001 by Underhill et al. and entitled PERSONAL WEAR ARTICLE WITH WETNESS INDICATOR, the entire disclosures of which are incorporated herein by reference, for additional details and alternative constructions of the wetness indicator 102.

In use, the wetness indicator 102 is generally soft, pliable and cloth-like when dry and has a stiffness generally similar to that of other portions of the pants 20, and more particularly the absorbent structure 44, making the presence of the wetness indicator generally imperceptible to the wearer prior to urination. The pliable wetness indicator 102 allows the thighs of the wearer to move freely and to readily compress the wetness indicator during normal movements. Upon the first insult of liquid body exudates, the wetness indicator 102 absorbs liquid, and the absorbent body 108 within the enclosure 106 begins to swell. This swelling of the absorbent body 108 applies a hydraulic pressure against the enclosure 106. The increased stiffness of the wetness indicator 102 provides a resistance to bending, folding, creasing, flexing, etc. of the pants 20, particularly in the crotch region 26, that may be readily perceived by the wearer. For example, the increased stiffness may resist movement by the wearer to bring the thighs of the wearer closer together. Such resistance against the inner thighs need not be large, but is suitably sufficient to at least gently resist leg movement of the inside of the legs of the wearer such that the wearer perceives the resistance, which the wearer eventually learns to associate with urination.

The combination of the tactile signal felt by the wearer upon stiffening of the wetness indicator 102 and the non-tactile signal provided by the interior active graphics 90 provides the aforementioned synergistic toilet training effect that results in a more beneficial toilet training experience. For example, upon wetting the toilet training pants 20, a child may first feel the effects of the wetness indicator 102 and be prompted to check the status of the graphic 90. Also, a caregiver who notices the wearer of the pants 20 is uncomfortable in the pants and experiencing resistance to movement may discuss with the child whether the graphic 90 has disappeared in an effort to advance the toilet training process.

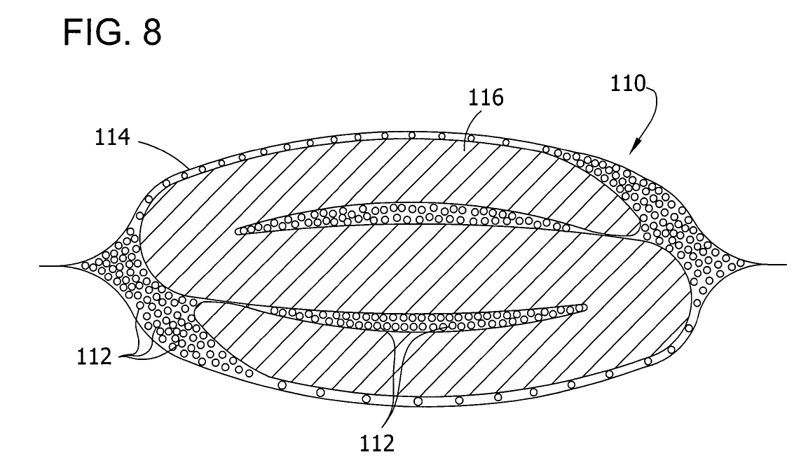

FIG. 8 illustrates an another embodiment of a suitable wetness indicator 110 similar to the wetness indicator 102 of FIGS. 5-7 but having a temperature change agent 112 that facilitates a tactile signal (e.g., a hot or cold sensation) against the wearer's skin to further alert the wearer that urination has occurred. The temperature change agent 112 is suitably responsive to contact with an aqueous solution, such as urine, to either absorb or release heat. The temperature change can be caused by either an absorption or a release of heat by the temperature change agent 112 to change the temperature of the urine and hence surrounding components of the pants to a temperature noticeable to the wearer. For example, an adsorption of heat by the temperature change agent 112 will provide a cool sensation against the wearer's skin while a release of heat by the temperature change agent will provide a warm sensation (e.g., warmer than the wearer's skin temperature) against the wearer's skin. Reference is made to aforementioned U.S. patent application Ser. No. 10/462,166, incorporated by reference herein, for additional information regarding the mechanism by which the temperature change sensation is accomplished.

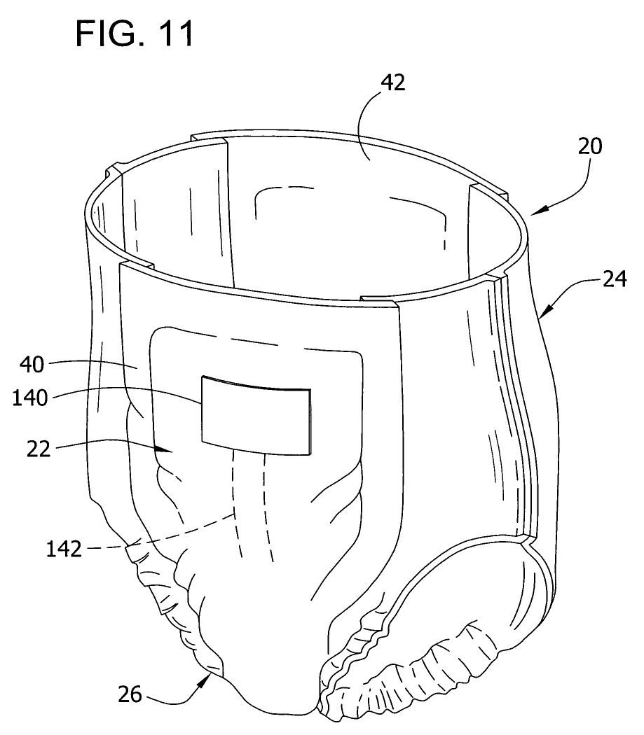

In the illustrated embodiment of FIG. 8, the temperature change agent 112 is in the form of endothermic material particles, disposed within the liquid permeable enclosure 114 of the wetness indicator 110. In use, as urine passes through the liquid permeable enclosure 114 of the wetness indicator 110 of FIG. 10, the urine comes into contact with and dissolves the temperature change agent 112 during or shortly after urination. The temperature change agent 112 absorbs heat from the urine upon dissolution and the cooled urine is then absorbed into the absorbent body 116. The wetness indicator 110 stiffens upon absorption of the cooled urine as described previously and applies pressure against the wearer's skin, such as against the wearer's inner thighs as described previously. The cooled wetness indicator 110 acts in the manner of a heat sink in thermally conductive contact with the wearer's skin (e.g., via the liner 42 and/or containment flaps 46 of the pants 20) to thereby draw heat from the wearer and provide a cool sensation to the wearer's skin. Positioning the cooled wetness indicator in thermally conductive contact with the wearer for a significant duration of time allows the temperature change resulting from the temperature change agent 112 to be more easily noticed by the wearer.

The swelling and stiffening of the wetness indicator 110 thus facilitates a more direct thermally conductive contact between the cooled wetness indicator and the wearer's skin. Use of the stiffening wetness indicator 110 to hold the cooled sensation against the wearer's skin reduces the ability of the wearer to escape thermally conductive contact with the heat sink (e.g.., the cooled and stiffened wetness indicator 45) by shifting their body position. It is also contemplated that temperature change agent 112 could be used in training pants without the stiffening wetness indicator 110 and remain within the scope of this invention.

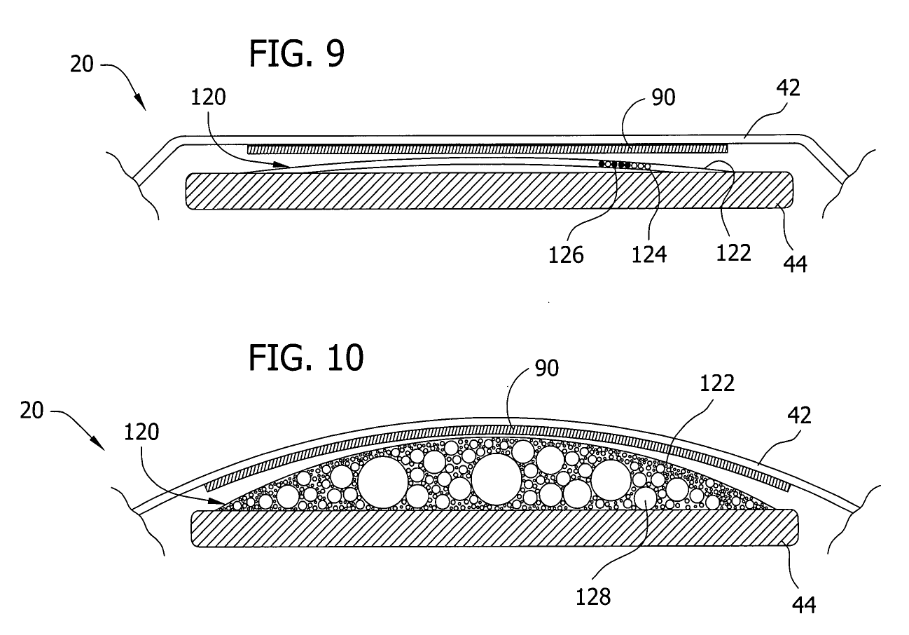

FIGS. 9 and 10 illustrate another alternative embodiment of the training pants 20 having an interior active graphic 90 and a tactile insult indicator in the form of a wetness indicator generally indicated 120 that comprises a system capable of generating a gas upon being wetted with urine. The wetness indicator 120 comprises a urine-permeable inflatable container 122 positioned between the bodyside liner 42 and the absorbent core 44. The urine-permeable inflatable container 122 comprises a gas producing system 124 and a surfactant 126. As used herein, the term “urine-permeable” refers to a porous material that is water-permeable due to the flow of water and other aqueous liquids, such as urine, through the pores.

As shown in FIG. 10, the gas that is produced in the container 122 upon wetting of the container interacts with one or more surfactants 126 and produces foam 128 that inflates the container and causes it to press the bodyside liner 42 against the skin of the wearer to alert the wearer to urination. As shown in FIG. 10, the inflated container 122 pushes against the bodyside liner 42 and distorts the bodyside liner to press against the skin of the wearer to alert the wearer to urination. Reference is made to assigned U.S. patent Ser. No. 10/822,930, filed Apr. 13, 2004 by Long et al. and entitled TOILET TRAINING ARTICLE CONTAINING FOAMING AGENT, the entire disclosure of which is incorporated herein by reference, for additional details and alternative constructions of the wetness indicator 120 containing foaming agent.

As shown in FIGS. 9 and 10, the article 20 comprises an active graphic 90 printed on the absorbent structure facing surface of the bodyside liner 42. The active graphic 90 along with the wetness indicator 120 comprising the foam producing container 122 provide the aforementioned synergistic toilet training effect between the tactile and non-tactile signals provided upon liquid insult.

It will be understood that multiple tactile insult indicators (e.g., wetness, pressure, temperature, stiffness, etc.) may be combined without departing from the scope of this invention. Other examples of suitable tactile sensation indicators are disclosed in co-assigned U.S. Pat. Nos. 5,797,892, 5,702,376 and 5,649,914, the entire disclosures of which are incorporated herein by reference.

It is also contemplated that the non-tactile signal may be other than the interior active graphic 90 discussed previously. For example, in one embodiment the non-tactile signal may comprise exterior active graphics 94 (FIG. 2). Exterior active graphics 64 suitable for use with the present invention are described in U.S. Pat. No. 6,297,424 issued Oct. 2, 2001 to Olson, et al. and U.S. Pat. No. 6,307,119 issued Oct. 23, 2001 to Cammarota et al., the entire disclosures of which are incorporated by reference herein. It is contemplated that portions of the exterior graphics 64 may be inactive, or permanent graphics as long as other portions of the graphics are active in response to liquid insult of the pants 20.

It is also contemplated that both interior and exterior graphics 90, 94 may be provided as non-tactile insult indicators. The interior graphic 90 may be related or unrelated to a graphic theme that may be defined by the exterior graphics 64 (FIG. 2). In aspects where the interior graphic 90 is related to a theme established by the exterior graphics 64, it provides an opportunity for the wearer and the caregiver to interact and can improve the toilet training experience. For example, in one aspect the theme established by the exterior graphics 64 can include a graphic that cannot complete some action or observation after a fading interior graphic 90 has disappeared. This can be used as a motivational basis for teaching the child that it is within their control to permit the activity to continue for as long as the child can go without wetting his or her pants.

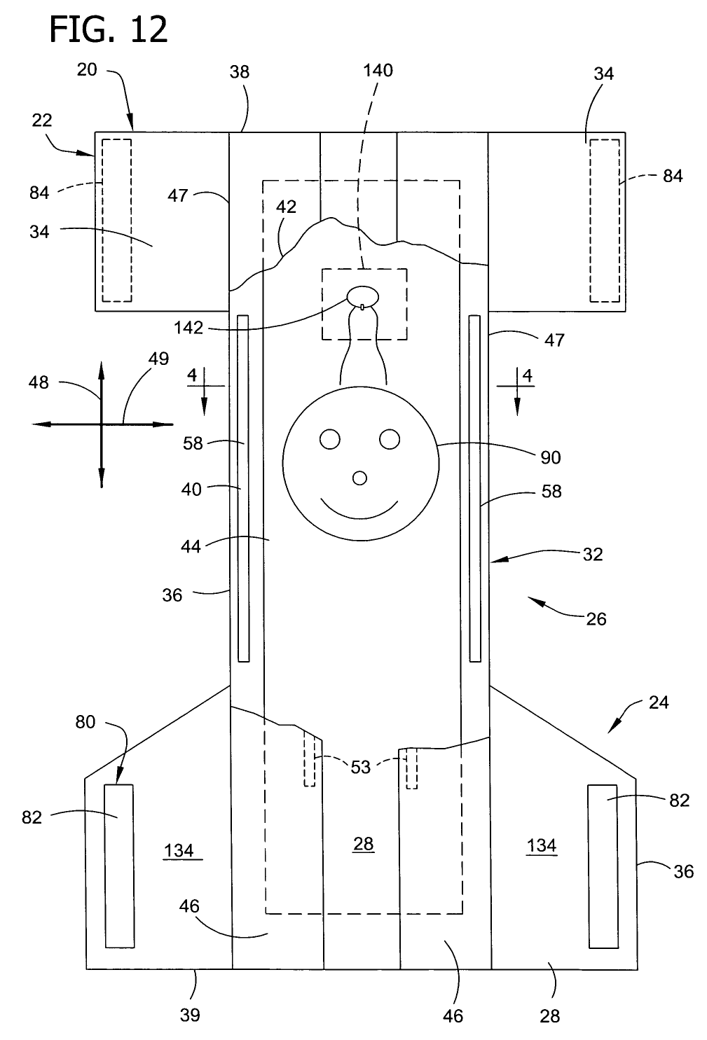

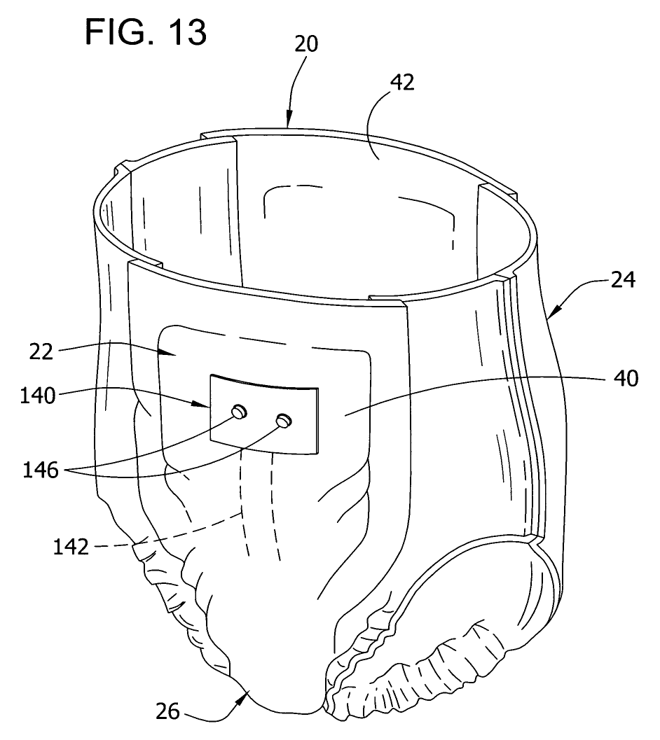

Other suitable non-tactile insult indicators for use in combination with a tactile insult indicator include an audible or remote vibratory wetness alarm 140 as shown in FIGS. 11 and 12 and described later herein, and a visual wetness alarm including lights as shown in FIG. 13 and also described later herein.

In other embodiments of the present invention, the pants 20 comprise at least one insult indicator that provides a signal that is perceptible within the interior of the pants upon liquid insult, and at least one insult indicator that provides a signal that is perceptible exterior of the pants upon liquid insult. For example, the embodiment illustrated in FIG. 4A, the training pants 20 comprises a first insult indicator in the form of exterior active graphics 64 and a second insult indicator in the form of an interior active graphic 90. The exterior active graphics 64 is preferably a disappearing graphic that provides a signal perceptible exterior of the article 20 upon the occurrence of a liquid insult. The exterior graphic 64 may be printed on the outer cover facing surface of the absorbent structure 44 as shown in FIG. 4A or any other layer (e.g., outer cover 40) of the pants 20 as long as the graphic is visible from the external surface 30 of the article. In the embodiment of FIG. 4A, the interior active graphic 90 is printed on the bodyside liner facing surface of the absorbent structure 44 but it is understood that the graphic could be otherwise positioned in the article as long as the graphic is visible from the interior surface 28 of the article. The training pants of FIG. 4A promote a synergistic training effect by providing an external signal in the form of exterior fading graphics 64 that alert the caregiver that an insult has occurred and providing a visual signal to the wearer in the form of the interior graphic 90 that confirms the occurrence of the insult.

In another embodiment, illustrated in FIGS. 11 and 12, toilet training pants 20 comprises a first insult indicator in the form of a wetness alarm 140 attached to the pants that produces an audible alarm when the article is wet and a second insult indicator in the form of an active interior graphic 90 that appears or disappears upon the occurrence of a liquid insult. In one embodiment the wetness alarm has a sensor 142 embedded in a layer (e.g., the absorbent structure 44) of the article 20 that senses when the article is wet. The audible signal produced by the wetness alarm is used to indicate to the caregiver and wearer of the article 20 when an accident has occurred. Reference is made to U.S. Pat. Nos. 5,341,127, 5,568,128, 6,384,728, and 6,583,722, incorporated by reference herein, for additional information regarding suitable wetness alarms.

In use, the wetness alarm 140 enhances the toilet training effect by providing audible indication to the caregiver which eliminates the need for frequent monitoring of the graphic 90 by the caregiver. In turn, the graphics 90 also provide a quick verification of the accuracy of the wetness alarm that may be checked by the caregiver and child to confirm the occurrence of an insult.

It is contemplated that the wetness alarm 140 may provide a vibratory response to wetness instead of, or in addition to, an rather than an audible response. The vibration may occur in the training pant 20 as a result of a signal from the sensor 142 attached or embedded in the article so that the vibration is felt by the wearer. Alternatively, the wetness alarm may generate a signal to a remote device (not shown) that may be held by or attached to a caregiver in charge of monitoring a toilet training child. Also, the wetness alarm 140 may be part of a personal care monitoring system as set forth in U.S. Pat. No. 6,384,728 or a bedwetting alarm described in U.S. Pat. No. 5,341,127, or any other system that provides an alarm to indicate a toilet training insult has occurred.

As shown in FIG. 13, the wetness alarm 140 may instead, or may additionally, incorporate lights 146 for indicating the status of the pants 20. For example, two lights 146 could be mounted on the alarm 140 with one light indicating that the article 20 is dry and another light indicating that the article is wet. The lights could be different colors (e.g., red and green) to provide easy indication to the caregiver of the status of the article. As shown in U.S. Pat. No. 5,568,128, the alarm 140 could be removably mounted on the article 20 or the alarm could be permanently attached or embedded in any layer of the article.

In use, the wetness alarm 140 with lights 146 enhances the toilet training effect by providing a visual signal to the caregiver which eliminates the need for frequent monitoring of the graphic 90 by the caregiver. Upon receiving the visual signal of the lights 146, the caregiver may confront the child regarding the wetness of the pants 20 and discuss with the child whether the release of body exudates has occurred. In turn, the interior graphics also provide a quick verification of the accuracy of the wetness alarm that may be checked by the caregiver and the wearer to confirm the occurrence of an insult.

As various changes could be made in the above constructions and methods, without departing from the scope of the invention, it is intended that all matter contained in the above description and shown in the accompanying drawings shall be interpreted as illustrative and not in a limiting sense.

When introducing elements of the invention or the preferred aspect(s) thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A method of toilet training a wearer of a disposable absorbent article, said method comprising:
activating an active graphic in response to liquid insult of the article, said active graphic being visually perceptible from within the interior of the article; and;
providing at least one other signal in response to liquid insult of the article, said at least one other signal being perceptible from at least one of the interior and the exterior of the article.

2. The method of claim 1 wherein said at least one other signal is visually perceptible exterior of the article.

3. The method of claim 2 wherein the step of providing said at least one other signal comprises activating an active graphic in response to liquid insult of the article, said active graphic being visually perceptible exterior of the article.

4. The method of claim 2 wherein said providing step comprises providing a light signal that is visually perceptible exterior of the article.

5. The method of claim 1 wherein said at least one other signal comprises an audible signal perceptible exterior of the article.

6. The method of claim 1 wherein said providing step comprises providing a vibratory signal remote from the article in response to liquid insult of the article.

7. The method of claim 1 wherein said providing step comprises providing a wetness sensation against the wearer's skin in response to liquid insult of the article.

8. The method of claim 1 wherein said providing step comprises providing a temperature change sensation against the wearer's skin in response to liquid insult of the article.

9. The method of claim 1 wherein said providing step comprises providing a pressure sensation against the wearer's skin in response to liquid insult of the article.

10. An absorbent article for toilet training a wearer of the article, the absorbent article having an inner surface that faces the article wearer and an outer surface opposite said inner surface, said article comprising:
an outer cover at least in part defining the outer surface of the article;
a liner in opposed relationship with the outer cover and at least in part defining the inner surface of the article;
an absorbent structure disposed between the liner and the outer cover;
a first insult indicator comprising an active graphic visually perceptible from the inner surface of the article and activated in response to liquid insult of the article; and
a second insult indicator responsive to the liquid insult of the absorbent article to provide a signal perceptible from at least one of the inner surface of the article and the exterior of the article.

11. The absorbent article of claim 10 wherein said second insult indicator is responsive to liquid insult of the article to provide a signal that is visually perceptible from exterior of the article.

12. The absorbent article of claim 11 wherein said second insult indicator comprises an active graphic visually perceptible from exterior of the article and activated in response to liquid insult of the article.

13. The absorbent article of claim 11 wherein said second insult indicator comprises at least one light disposed on the article and visually perceptible from exterior of the article.

14. The absorbent article of claim 10 wherein said second insult indicator comprises an audible alarm activated in response to liquid insult of the article.

15. The absorbent article of claim 10 wherein said second insult indicator comprises a vibration alarm activated in response to liquid insult of the article.

16. The absorbent article of claim 10 wherein said second insult indicator is responsive to liquid insult of the article to provide a wetness sensation against the wearer's skin.

17. The absorbent article of claim 16 wherein said second insult indicator comprises a pledget comprised at least in part of absorbent material and being disposed between the absorbent structure and the liner.

18. The absorbent article of claim 17 wherein said pledget comprises coform material.

19. The absorbent article of claim 17 wherein the pledget provides a wetness sensation against the article wearer's skin for approximately 15 minutes after liquid insult of the article.

20. The absorbent article of claim 10 wherein said second insult indicator provides a temperature change sensation to the article wearer's skin in response to liquid insult of the article.

21. The absorbent article of claim 10 wherein said second insult indicator has a first stiffness prior to liquid insult of the article and a second stiffness greater than said first stiffness in response to liquid insult of the absorbent article.

22. The absorbent article of claim 10 wherein said second insult indicator applies a pressure sensation against the article wearer's skin in response to liquid insult of the article.

23. An absorbent article for toilet training a wearer of the article, the absorbent article having an inner surface that faces the article wearer and an outer surface opposite said inner surface, said article comprising:
an outer cover at least in part defining the outer surface of the article;
a liner in opposed relationship with the outer cover and at least in part defining the inner surface of the article;
an absorbent structure disposed between the liner and the outer cover;
a first insult indicator responsive to a liquid insult of the absorbent article to provide a non-tactile signal perceptible from the inner surface of the article; and
a second insult indicator responsive to the liquid insult of the absorbent article to provide a tactile sensation to the wearer.

24. The absorbent article of claim 23 wherein said first insult indicator is responsive to liquid insult of the article to provide a signal that is visually perceptible from the inner surface of the article.

25. The absorbent article of claim 24 wherein said first insult indicator comprises an active graphic visually perceptible from the inner surface of the article.

26. The absorbent article of claim 23 wherein said first insult indicator comprises an audible alarm activated in response to liquid insult of the article.

27. The absorbent article of claim 23 wherein said second insult indicator is responsive to liquid insult of the article to provide a wetness sensation against the wearer's skin.

28. The absorbent article of claim 27 wherein said second insult indicator comprises a pledget comprised at least in part of absorbent material and being disposed between the absorbent structure and the liner.

29. The absorbent article of claim 28 wherein said pledget comprises coform material.

30. The absorbent article of claim 28 wherein the pledget provides a wetness sensation against the article wearer's skin for approximately 15 minutes after liquid insult of the article.

31. The absorbent article of claim 23 wherein said second insult indicator provides a temperature change sensation to the article wearer's skin in response to liquid insult of the article.

32. The absorbent article of claim 23 wherein said second insult indicator has a first stiffness prior to liquid insult of the article and a second stiffness greater than said first stiffness in response to liquid insult of the absorbent article.

33. The absorbent article of claim 23 wherein said second insult indicator applies a pressure sensation against the article wearer's skin in response to liquid insult of the article.

34. The absorbent article of claim 23 wherein said second insult indicator comprises a vibration alarm activated in response to liquid insult of the article to provide a vibratory signal within the absorbent article.
